(19) United States
(12) Patent Application Publication
(10) Pub. No.: US 2024/0316242 A1
(43) Pub. Date: Sep. 26, 2024

(54) Elastic Sutures and Methods of Making and Use

(71) Applicant: ARTHREX, INC., Naples, FL (US)

(72) Inventors: Shaun G. LEBLANC, Naples, FL (US); Thomas DOONEY, Jr., Naples, FL (US); Michael SCHMIDT, Wessling (DE); David J. GOODNEY, Naples, FL (US); Okan ALA, Naples, FL (US)

(73) Assignee: ARTHREX, INC., Naples, FL (US)

(21) Appl. No.: 18/187,733

(22) Filed: Mar. 22, 2023

(51) Int. Cl. A61L 17/14

(52) U.S. Cl. CPC A61L 17/145

(57) ABSTRACT

Surgical elastic constructs, assemblies, and methods of manufacturing, as well as methods for tissue repairs are disclosed. An elastic suture includes an elastic component to allow for knotless suture constructs and eliminate the need to tie knots as needed with standard suture. The elastic suture can include a silicon core and a polyester/UHMWPE jacket. The elastic suture can have a non-regular braid, weave, or knit pattern. The elastic suture can be manufactured with varying and controlled elasticity based on a particular application.

### BACKGROUND

The disclosure relates to the field of surgery and, more specifically, to surgical suture constructs and methods of making, and tissue repairs for reconstructive surgeries.

### SUMMARY

Surgical elastic flexible couplers, methods of making, and methods of tissue repairs are disclosed.

A surgical flexible coupler can be an elastic suture. An elastic suture includes an elastic component to allow for knotless suture constructs and to eliminate the need to tie knots as needed with standard suture. An elastic suture can include a silicon core and a polyester/UHMWPE jacket. An elastic suture can have a non-regular braid, weave, or knit pattern, or combinations thereof. An elastic suture can be manufactured with varying and controlled elasticity based on a particular application.

Methods of knotless, tensionable tissue repairs with elastic flexible couplers are also disclosed. An elastic suture can provide knotless tissue fixation, to allow for dynamic repairs with the ability to move and rebound when necessary. An elastic suture can allow for suture stretching to keep a knot secure. An elastic suture can allow for better bunching and fixation for anchor sheaths. An elastic suture can provide an overall more secure locking mechanism over the repair area for any tensionable knotless anchor.

### DESCRIPTION OF THE DRAWINGS

FIG. 1 illustrates a front view of a flexible elastic coupler.

FIG. 2 is a side view of the flexible elastic coupler of FIG. 1.

FIG. 3 illustrates a perspective view of another flexible elastic coupler.

FIG. 4 illustrates a front view of another flexible elastic coupler.

FIG. 5 is an enlarged view of an elastic/non elastic junction of the flexible elastic coupler of FIG. 4.

FIG. 6 illustrates a front view of another flexible elastic coupler.

FIG. 7 is an enlarged view of an elastic/non elastic junction of the flexible elastic coupler of FIG. 6.

FIG. 8 illustrates a flexible elastic coupler in a relaxed position.

FIG. 9 illustrates the flexible elastic coupler of FIG. 8 in a stretched position.

FIG. 10 illustrates another flexible elastic coupler.

FIGS. 11 and 12 illustrate steps of a tissue repair (knot tying) with a flexible elastic coupler.

FIGS. 13 and 14 illustrate a tissue repair (internal brace) with a flexible elastic coupler.

FIGS. 15-18 illustrate subsequent steps of the tissue repair (internal brace) of FIG. 14.

FIGS. 19 and 20 illustrate surgical constructs with a flexible elastic coupler.

FIG. 21 illustrates another flexible elastic coupler.

FIGS. 22-25 illustrate subsequent steps of a tissue repair (glenoid labrum repair) with a flexible elastic coupler.

FIGS. 26-35 illustrate subsequent steps of a tissue repair (glenoid labrum repair) with a flexible elastic coupler.

FIGS. 36-43 illustrate subsequent steps of a tissue repair (fracture reduction and fixation with tape cerclage) with a flexible elastic coupler.

FIG. 44 illustrates a side view of a repair with a flexible elastic coupler.

FIG. 45 illustrates a side view of another repair with a flexible elastic coupler.

FIGS. 46-50 illustrate steps of another tissue repair (racking half hitch) with a flexible elastic coupler.

### DETAILED DESCRIPTION

The disclosure provides surgical flexible elastic couplers, assemblies, constructs, and methods for manufacturing and tissue repairs and reconstructions.

A flexible coupler can be a surgical elastic suture that includes an elastic component which allows for a knotless suture construct and eliminates the need to tie knots as needed with standard suture.

Round and/or tape suture can be manufactured via braiding, weaving and/or knitting processes with an elastic component incorporated in them. In an embodiment, elastic suture can include an elastane core and a polyester/UHMWPE jacket, or a combination of elastic, polyester and UHMWPE in the core and/or jacket. The elastic component provides the elasticity while the other components provide the strength and limit the elongation of the suture. When the suture relaxes, the elastic component contracts and the other components compress giving the suture its elastic property. When the suture is relaxed, the core relaxes and the jacket compresses around giving the suture its elastic property as well as the limit from the jacket. When the suture is under tension, the body of the suture shrinks/narrows and, when released, it expands to lock in the place. The elastic suture can be completed with varying and controlled elasticity based on the application. Complex suture structures can be constructed/manufactured by providing non-regular braid patterns, for example, links, loops, splits, different size holes in the structure, downsized, takeout, bi-furcation, tri-furcation, quad-furcation, etc. The non-regular braid patterns can be provided by themselves or in combination with regular patterns.

Methods of manufacturing elastic flexible couplers are also disclosed. An exemplary method of making an elastic flexible coupler includes the step of incorporating an elastic component into the coupler via braiding, weaving, and/or knitting. The method can include the step of providing a non-regular braid pattern into the elastic coupler.

Methods of tissue repairs with elastic flexible couplers are also disclosed. An exemplary method includes inter alia the steps of: (i) incorporating an elastic component into a flexible coupler via braiding, weaving and/or knitting to form an elastic flexible coupler; and (ii) employing the elastic coupler to one or more tissue repair applications. The one or more tissue repair applications can include knot tying; soft tissue repair; internal brace repair; cerclage repair; and racking half hitch repair, among many others. The one or more tissue repair applications can include one or more knotless cerclage and/or tensionable knotless mechanisms.

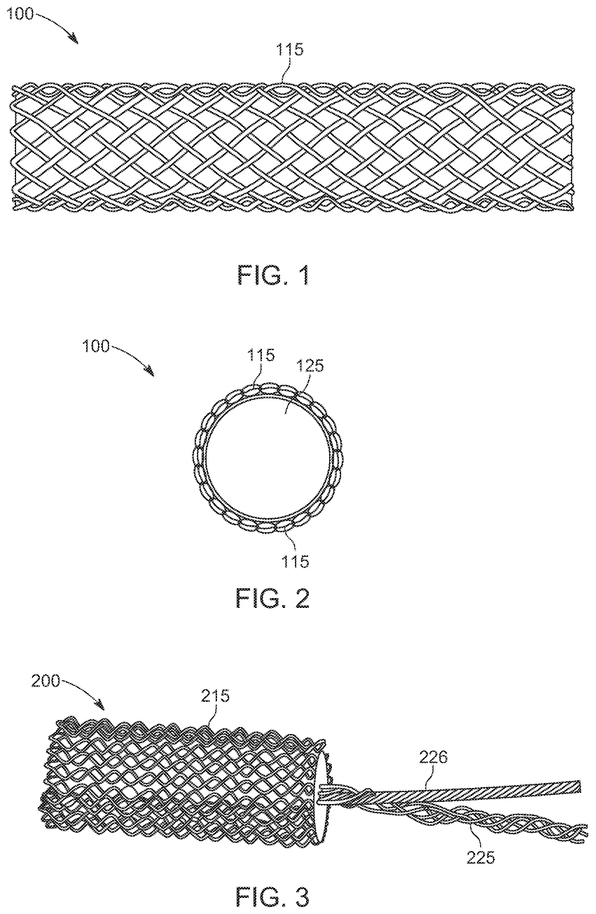

Referring now to the drawings, where like elements are designated by like reference numerals, FIGS. 1-10 illustrate exemplary surgical elastic flexible couplers 100, 200, 300, 400, 500, 600 (elastic sutures 100, 200, 300, 400, 500, 600; round suture 100, 200, 300, 400, 500, 600; round suture/tape constructs 100, 200, 300, 400, 500, 600; round-to-flat sutures 100, 200, 300, 400, 500, 600; complex sutures 100, 200, 300, 400, 500, 600; elastic couplers 100, 200, 300 400, 500, 600) including at least one elastic component 125, 215, 225, 315, 325, 415, 425, 515, 525 (elasticity component; elastomer; elastic fiber) of the present disclosure. FIGS. 11-50 illustrate steps of exemplary tissue repairs 101, 201, 301, 401, 501, 601 with elastic flexible couplers 100, 200, 300, 400, 500, 600.

### Elastic Suture—Types and Manufacturing

As detailed below, elastic flexible couplers in the form of elastic round and/or flat tape sutures can be manufactured via braiding weaving and/or knitting processes, or combination of these processes, with an elastic component in them. The elastic component provides the elasticity while the other components provide the strength and limit the elongation of the suture. When the flexible coupler (suture) is relaxed, the elastic contracts and the other components compress giving the flexible coupler (suture) its elastic property. The elastic flexible coupler (suture) can be manufactured with varying and controlled elasticity based on the application. Complex suture structures manufactured by the present disclosure include regular and non-regular braid patterns (i.e., links, loops, splits, different size holes in the structure, downsized, takeout, bi-furcation, tri-furcation, quad-furcation, multiple-furcation, etc.). The elastic component provides flexibility to the flexible coupler in at two different directions: a longitudinal direction (i.e., elongation in a direction about parallel to the longitudinal axis of the suture construct); and another direction, for example, a direction non-parallel to the longitudinal axis of the flexible construct.

FIG. 1 illustrates an embodiment of an elastic flexible coupler 100 in the form of elastic suture 100 having an elastic tubular structure. In an exemplary embodiment, elastic suture 100 has a braided structure, i.e., provided with a braid or jacket 115 that covers an elastic core 125. Elastic core 125 can consist essentially of elastomeric fibers. Elastic core 125 can consist essentially of elastane material. Braid or jacket 115 can consist essentially of polyester and ultra-high molecular weight polyethylene (UHMWPE).

Elastic suture 200 of FIG. 3 is a warp knitted suture constructed with a parallel, twisted and/or braided, knitted, woven elastane material in the warp or weft direction, with one or multiple filaments with/without non-elastane textile material composing the balance of the construction. Elastic behavior can be manipulated via elastic material insertion or removal within the same suture which creates elastic/non-elastic segments within the body. Elastic suture 200 includes an elastic braid or jacket 215 and a core formed of elastic material 225 (elastic segment 225; elastic fibers 225) and non-elastic material 226 (non-elastic segments 225; non-elastic fibers 225). At least one of the elastic material 225 (elastic segment 225; elastic fibers 225) can be elastane.

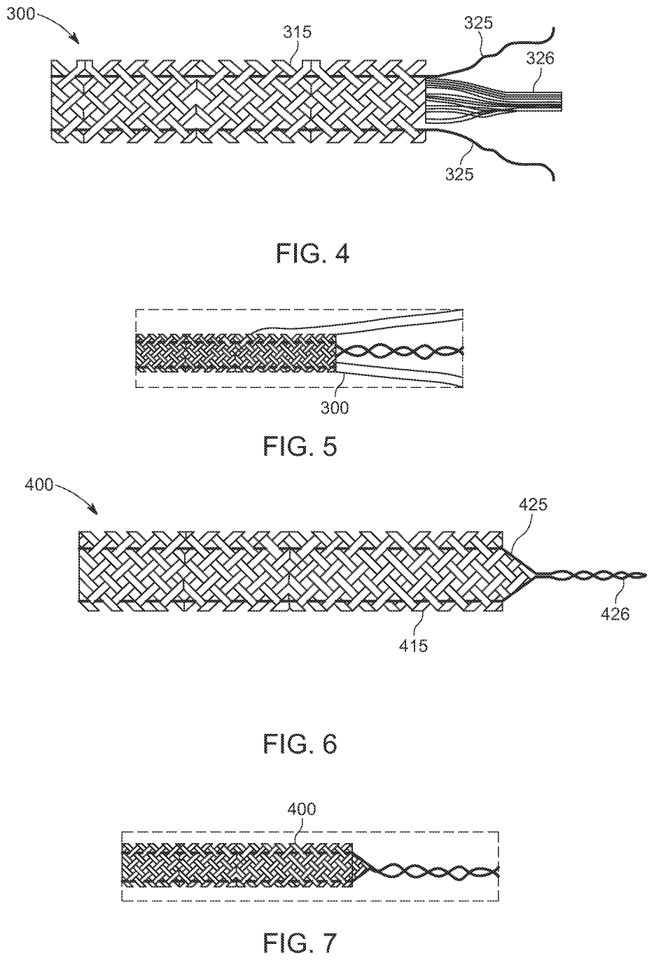

FIGS. 4-10 illustrate additional round, flat and round-to-flat and complex sutures 300, 400, 500. Suture 300 of FIGS. 4 and 5 is an exemplary elastic flexible coupler in the form of elastic flat (tape) with an elastic flat body 315 and with two elastic round tails 325 incorporated within the body 315 and with one non-elastic round tail 326. As shown in FIG. 4, the two elastic round tails 325 extend in a direction about parallel to a longitudinal axis of the body 315.

Suture 400 of FIGS. 6 and 7 is an exemplary tubular and flat elastic suture with an elastic flat body 415 and with a tubular elastic tail 426 (also incorporating two elastic round tails 425 incorporated within the body 415 and extending in a direction about parallel to a longitudinal axis of the body 415).

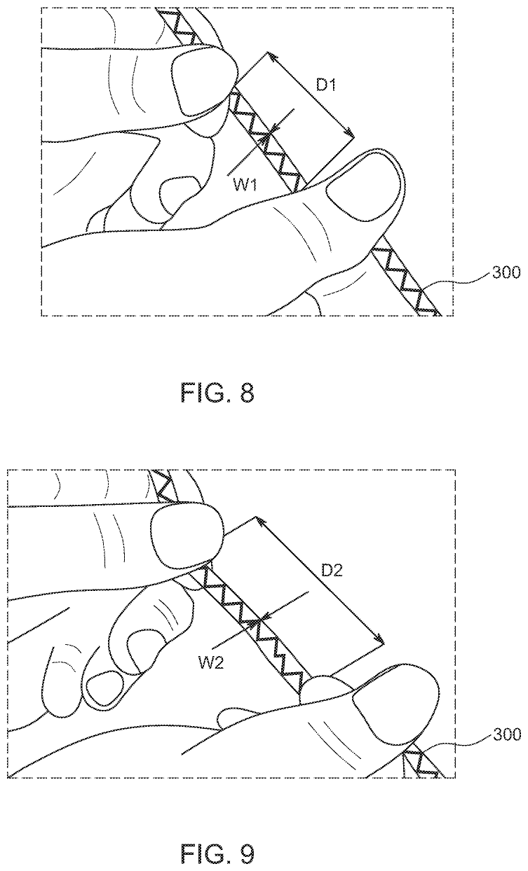

FIGS. 8 and 9 illustrate the flexibility and elasticity of elastic suture of the present disclosure. Length D2 of exemplary elastic coupler 300 of FIG. 9 is greater than length D1 of elastic coupler 300 of FIG. 8. Width W2 of elastic coupler 300 of FIG. 9 is smaller than width W1 of elastic coupler 300 of FIG. 8

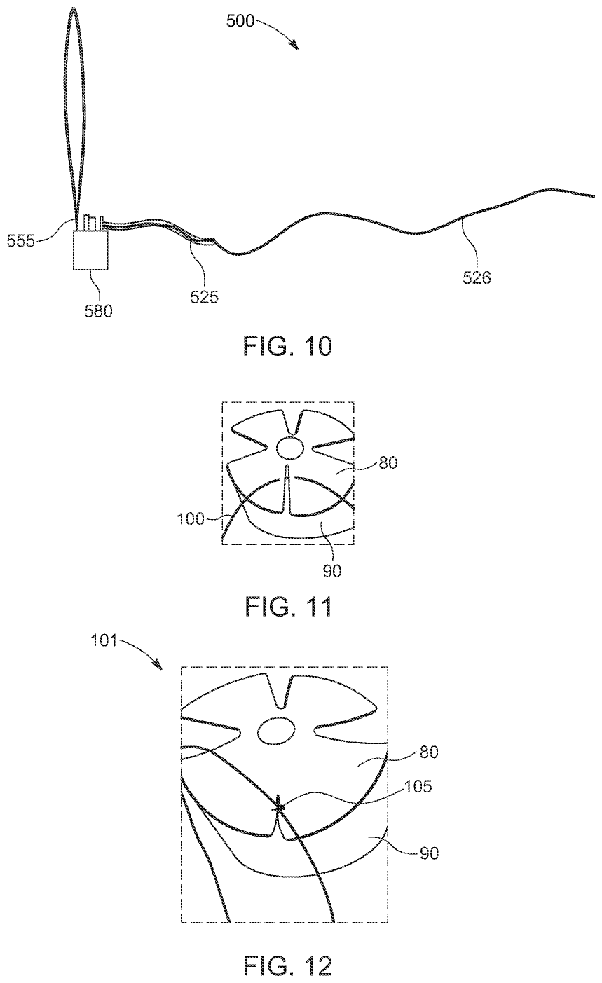

FIG. 10 illustrates yet another exemplary elastic flexible coupler 500 of the present disclosure. Flexible coupler 500 is an elastic suture 500 that includes a first portion 525 (first segment 525 or first length 525) adjacent to a second portion 526 (second segment 526 or second length 526). In one embodiment, one of the first and second portions 525, 526 is elastic and the other of the first and second portions 525, 526 is non-elastic. In one embodiment, suture segment 525 is elastic. In one embodiment, suture segment 526 is non-elastic. In one embodiment, portions or segments of elastic and non-elastic segments can alternate in a pattern along the length of the suture. In one embodiment, portions or segments of elastic and non-elastic segments can alternate in a random manner along the length of the suture. In yet additional embodiments, regular patterns (of elastic and non-elastic segments) can alternate with randomly provided segments (elastic and non-elastic segments) along the length of the suture.

Elastic suture 100, 200, 300, 400, 500 can be a woven suture constructed with a parallel, twisted and/or braided, knitted, woven elastane material in the warp or weft direction, with one or multiple filaments with/without non-elastane textile material composing the balance of the construction. Elastic behavior can be manipulated via elastic material insertion or removal within the same suture which creates elastic/non-elastic segments within the body.

In another embodiment, elastic suture 100, 200, 300, 400, 500 can be a warp knitted suture constructed with a parallel, twisted and/or braided, knitted, woven elastane material in the structure with one or multiple filaments with/without non-elastane textile material composing the balance of the construction. Elastic behavior can be manipulated via elastic material insertion or removal within the same suture which creates elastic/non-elastic segments within the body.

In another embodiment, elastic suture 100, 200, 300, 400, 500 can be a weft knitted suture constructed with a parallel, twisted and/or braided, knitted, woven elastane material in the structure with one or multiple filaments with/without non-elastane textile material composing the balance of the construction. Elastic behavior can be manipulated via elastic material insertion or removal within the same suture which creates elastic/non-elastic segments within the body.

Round/flat/round-to-flat/complex suture 100, 200, 300, 400, 500 can be constructed with a parallel or twisted elastane core(s) with one or multiple filaments with/without a non-elastane textile material jacket.

Round/flat/round-to-flat/complex suture 100, 200, 300, 400, 500 can be constructed with a parallel or twisted elastane warp(s) with one or multiple filaments with/without a non-elastane textile material composing the balance of the construction.

Round/flat/round-to-flat/complex suture 100, 200, 300, 400, 500 can be constructed with a parallel or twisted elastane warp(s) and core(s) with one or multiple filaments with/without a non-elastane textile material composing the balance of the construction.

Round/flat/round-to-flat/complex suture 100, 200, 300, 400, 500 can be constructed with elastane material in the jacket of the braid with/without a non-elastane textile material composing the balance of the construction.

Round/flat/round-to-flat/complex suture 100, 200, 300, 400, 500 can be constructed with elastane material in the jacket and in the warp(s) of the braid with/without a non-elastane textile material composing the balance of the construction.

Round/flat/round-to-flat/complex suture 100, 200, 300, 400, 500 can be constructed with Elastane material in the jacket and in the core(s) of the braid with/without a non-elastane textile material composing the balance of the construction.

Round/flat/round-to-flat/complex suture 100, 200, 300, 400, 500 can be constructed with a parallel or twisted elastane, jacket, warp(s), and core(s) with one or multiple filaments with/without a non-elastane textile material composing the balance of the construction.

Round/flat/round-to-flat/complex suture 100, 200, 300, 400, 500 can be constructed in such a way that the elastic material can be diverted, inserted, or removed at desired locations regardless of the suture configuration which creates elastic/non-elastic segments within the same suture.

Round and/or tape style suture 100, 200, 300, 400, 500 can be manufactured via braiding, weaving and/or knitting processes with an elastic component incorporated in them. In an embodiment, surgical elastic suture 100, 200, 300, 400, 500 can include an elastane core and a polyester/UHMWPE jacket, or a combination of elastic, polyester and UHMWPE in the core and/or jacket.

The elastic component provides the elasticity while the other components provide the strength and limit the elongation of the suture. When the suture relaxes, the elastic component contracts and the other components compress giving the suture its elastic property. When the suture is relaxed, the core relaxes and the jacket compresses around giving the suture its elastic property as well as the limit from the jacket. When the suture is under tension, the body of the suture shrinks/narrows and when released it expands to lock in the place. The elastic suture can be completed with varying and controlled elasticity based on the application. Complex suture structures can be constructed/manufactured by providing non-regular braid patterns, for example, links, loops, splits, different size holes in the structure, downsized, takeout, bi-furcation, tri-furcation, quad-furcation, etc.

### Elastic Suture 100, 200, 300, 400, 500—Applications

Knot tying—the knot is under tension and the tails are relaxed causing them to bunch up and prevent the knot from backing out. The tails are difficult to slide into the tension of the knot providing more knot strength and fewer knots needed to secure tissue.

Internal Brace—using an elastic suture/tape with a certain amount of elasticity built into the construct for flexibility offers a definite stop where the elastic portion is fully extended and the polyester/UHMWPE acts as the stopping point.

Soft Tissue Repair—the suture provides consistent longer lasting pressure on the tissue and can provide a dynamic repair that moves with the soft tissue. This aspect also provides a self-tensioning effect if the repair becomes loose.

Knotless Cerclage/Tensionable Knotless Mechanisms—the Chinese Finger Trap is elastic providing more holding strength and less slippage in the mechanism. Another option for the tensionable knotless is to have the repair suture in elastic, to allow for additional holding strength as well as dynamic repair to the tissue.

Racking Half Hitch—the elastic suture designed with a rough sheath can hold without the need to tie additional half hitches to lock the racking hitch.

Below we address the above-listed applications and/or constructs in more detail:

### Soft Tissue Repair/Knotless Side to Side Repair

Soft tissue repairs such as knotless side to side repairs can be conducted with any and/or all flexible elastic couplers 100, 200, 300, 400, 500. Reference is now made to FIG. 10 (illustrating exemplary elastic suture 500). The suture provides consistent longer lasting longer pressure on the tissue and can provide a dynamic repair that moves with the soft tissue. This aspect also provides a self tensioning effect if the repair becomes loose. Using a splicing technique with the elastic suture, allows the suture 500 to create a knotless repair where the suture narrows when pulled into the repair location and then relaxes causing the suture to lock into place.

The suture construct 500 can be made up of round or flat tape style suture with a poly/UHMWPE and elastic combination. The elastic portion 525 of the suture can be spliced back through itself to create a knotless repair, locking splice 555.

While tensioning, the elastic properties of the suture elongate and pass through the locking splice 555. When released at the desired tension, the elastic suture 525 relaxes and creates additional holding strength and less slippage in the construct.

The jacket of the elastic portion 525 of the suture 500 can be configured differently to increase holding strength. A suture passing flag/link/device 580 can hold the locking splice 555 open for ease of converting. The tail/tails 526 can be passed through the locking splice 555 with the passing device to complete a knotless repair. The construct can be tightened down to secure the tissue. The elastic suture 525 stretches/narrows during tension/tightening and then relaxes to capture/secure in place.

### Knot Tying

Reference is now made to FIGS. 11 and 12, which, for simplicity illustrate only elastic suture 100, although any of elastic flexible couplers 100, 200, 300, 400, 500 can be employed for knot tying. Elastic suture 100 is passed through first tissue 80 adjacent second tissue 90 (for example, soft tissue 80 adjacent bone 90) forming knot 105 of repair 101. The knot is under tension and the tails are relaxed causing them both to bunch up and prevent the knot from backing out. The tails are difficult to slide into the tension of the knot providing more knot strength and fewer knots needed to secure tissue 80.

Elastic suture 100 can be passed through tissue 80 with a needle or inserter. One or more knots of choice (desired knots) are tied with the suture tails. Elastic suture 100 stretches when tying the knot and then relaxes when not under tension. Exemplary knot 105 is locked in place and not allowed to loosen (to come loose), resulting in smaller and tighter knot stack(s).

### Internal Brace

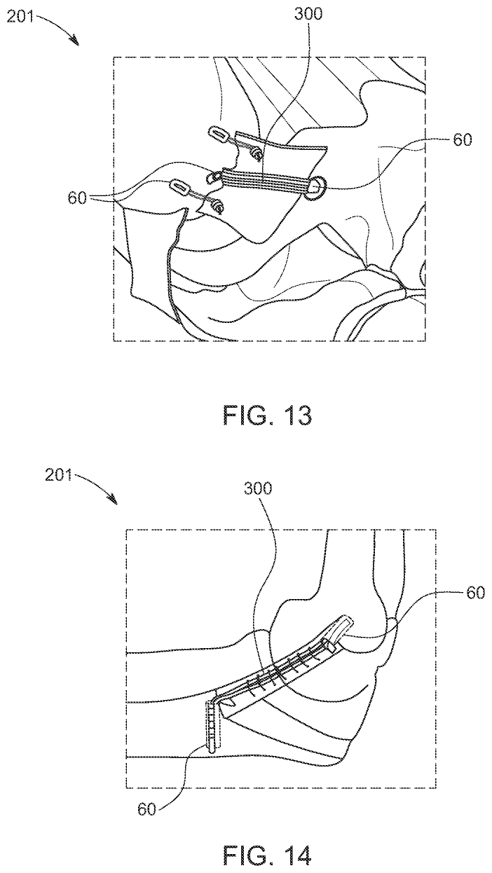

Reference is now made to FIGS. 13-18, which, for simplicity illustrate only elastic suture 300, although any of elastic suture 100, 200, 400, 500 can be employed for internal brace repair 201 (FIGS. 13 and 14). Using an elastic suture/tape 300 that has a certain amount of elasticity built into the construct for flexibility offers a definite stop where the elastic portion is fully extended and the non-elastane material (such as, for example, UHMWPE/polyester combination) acts as the stopping point. As shown in FIGS. 13 and 14, elastic suture/tape 300 is attached to exemplary fixation devices 60 (for example, two suture anchors 60 inserted into bone 90).

Fixation devices 60 can be any anchors, for example, knotted anchors, knotless anchors, or all-suture anchors or any devices that confer secure attachment and fixation of soft tissue 80 over bone 90 and that allow sliding of elastic suture within a body of fixation device. Fixation device 60 can be a knotless suture anchor such as a two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of both of which are fully incorporated by reference in their entirety herein.

An elastic tape-style suture, round or flat, can be similar in size and shape to a suture tape such as FiberTape® suture tape. The elastic tape is designed and manufactured to allow for specific amount of stretch and/or elasticity for a dynamic repair. When fully extended, the elastic suture 300 has a positive stop from the non-elastic portion of the suture, providing additional strength.

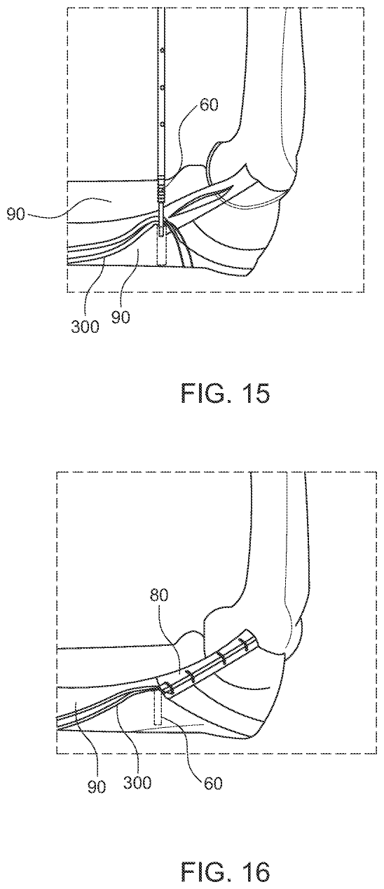

FIGS. 15-18: Elbow UCL Repair with the UCL Elastic InternalBrace™ System

Medial ulnar collateral ligament (UCL) repair of the elbow can be conducted with any of the elastic flexible couplers 100, 200, 300, 400, 500.

FIG. 15: Load a 3.5 mm PEEK SwiveLock® anchor 60 with elastic coupler 300 (an elastic FiberTape® suture tape or an elastic collagen-coated FiberTape® suture tape) and a FiberWire® suture repair stitch. Insert anchor 60 into the socket until the anchor body makes contact with bone 90. Hold the thumb pad and rotate the driver handle clockwise until the anchor 60 is flush with the bone 90.

FIG. 16: Repair the native ligament 80 back to the sublime tubercle using the FiberWire® suture. Cut the sutures and repair any incised portion of the ligament with side-to-side stitches using the FiberWire® suture.

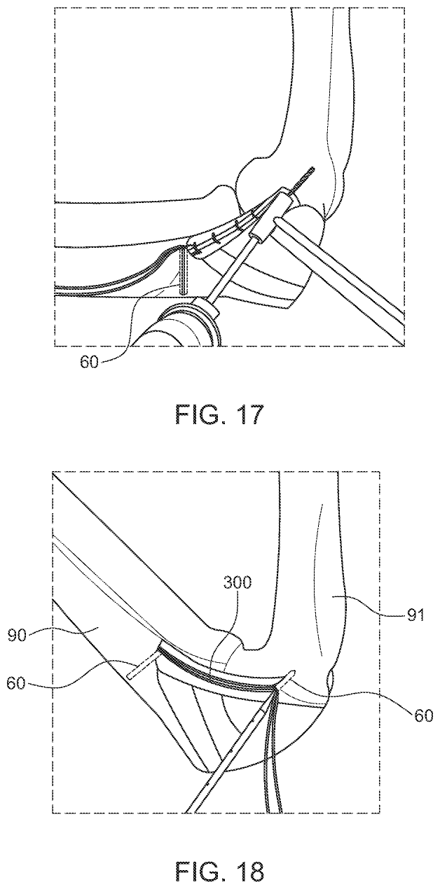

FIG. 17: Place the drill guide on the proximal origin of the UCL at the medial epicondyle. Drill a socket using the 2.7 mm drill. Tap the socket with the red-handled tap.

FIG. 18: Load the tails of FiberTape® suture tape 300 from the distal anchor into a 3.5 mm PEEK SwiveLock® anchor 60. Place the arm in about 35°-45° of flexion. Insert the anchor 60 into the socket until the anchor body makes contact with bone 91. Take the elbow through full range of motion to check for adequate tensioning. Hold the thumb pad and rotate the driver handle clockwise until the anchor 60 is flush with the bone 91. Use the remaining FiberWire® suture to sew the FiberTape® suture tape 300 to the underlying ligament to complete the repair 201 (shown in FIGS. 13 and 14).

### Elastic Tensionable Knotless Mechanisms

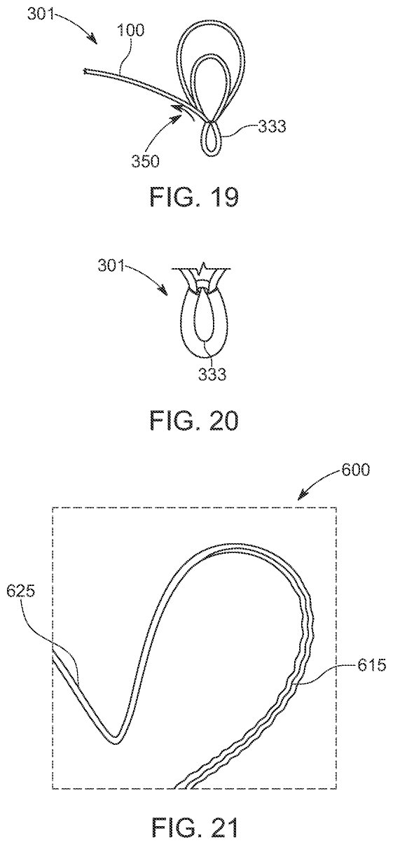

Reference is now made to FIGS. 19 and 20. Exemplary tensionable knotless mechanism (TKL) 301 can incorporate any of elastic sutures 100, 200, 300, 400, 500. TKL 301 is elastic and provides more holding strength and less slippage in the mechanism. Also, the repair suture in elastic allows for additional holding strength as well as dynamic repair to the tissue. Sheath 333 of the knotless anchor 350 can be made from elastic providing better fixation in bone.

In additional embodiments, both the repair suture 100 and the TKL mechanism 301 can be made with elastic suture. While tensioning the repair suture, the elastic properties of the suture would elongate and pass through the TKL mechanism and when released and the desired tension the elastic suture would relax and create additional holding strength and less slippage in the construct. This aspect can be done in both hard and soft anchor constructs.

In yet additional embodiments, sheath 333 of exemplary FiberTak® soft anchor 350 can be made with elastic material. This aspect allows the sheath to stretch and elongate on insertion and then expand after final anchor setting.

FIG. 21 illustrates a perspective view of elastic flexible coupler 600 in the form of an elastic repair suture with a tapered tail. Elastic coupler 600 includes an elastic tape portion 615 and a tapered tail 625. Tapered tail 625 can be elastic or non-elastic. Tape portion 615 can be elastic throughout and along its whole length. Tape portion 615 can be partially elastic along its length, i.e., can contain elastic segments and non-elastic segments. As detailed above, the elastic and non-elastic segments can alternate and be provided in a pattern.

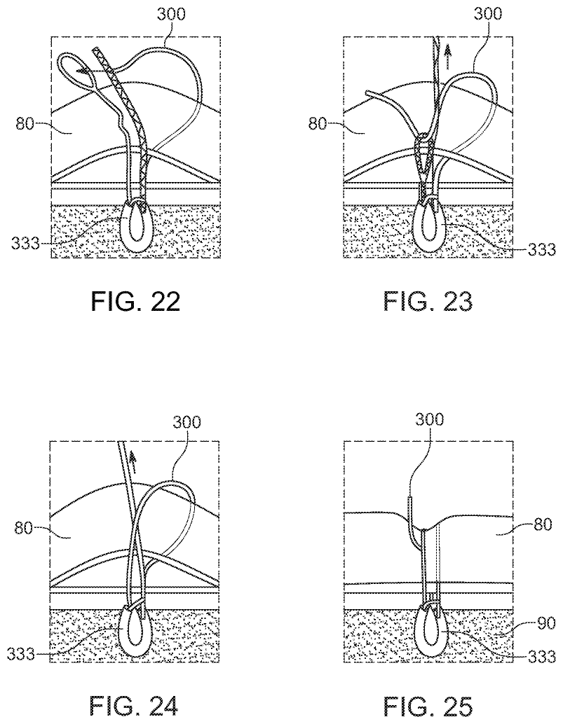

FIGS. 22-25 illustrate a series of subsequent steps of repair of labrum 80 using knotless, self-locking, tensionable soft anchor 350 of FIG. 19 employed with any elastic coupler 100, 200, 300, 400, 500, 600.

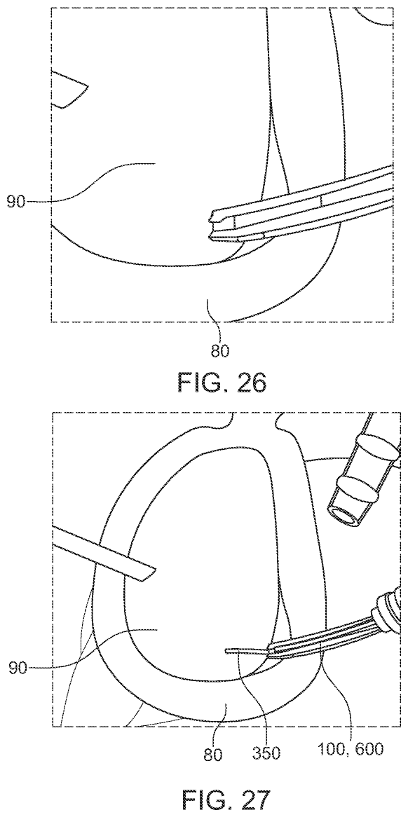

FIGS. 26-35: Elastic Knotless FiberTak® Soft Anchor for Glenoid Labrum Repair (with Knotless Simple Stitch or Knotless Mattress Stitch)

The embodiment below will be described with reference to a knotless FiberTak® soft anchor (for example, a knotless 1.8 FiberTak® soft anchor) which includes any of the elastic couplers 100, 200, 300, 400, 500, 600 and provides advanced self-locking technology with simple steps of passing, cinching, and cutting. An exemplary embodiment of a knotless FiberTak® soft anchor which includes any of the elastic couplers 100, 200, 300, 400, 500, 600 is knotless, self-locking, tensionable soft anchor 350.

FIG. 26: Mobilize labrum 80 and pass the spear through the cannula and place it on the glenoid rim 90. Create a bone socket for the anchor by advancing the drill through the spear until the collar contacts the handle of the spear.

FIG. 27: Insert anchor 350 through the spear and into bone 90 by gentle impaction until the inserter handle is flush with the back of the spear.

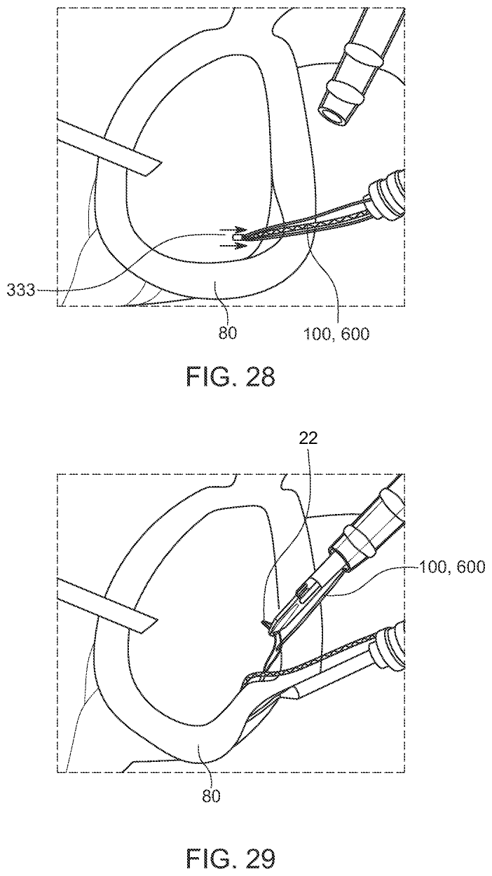

FIG. 28: Remove the inserter handle and spear, then pull on all three suture tails to confirm the anchor is set in the cortical bone.

FIG. 29: Retrieve exemplary-only elastic repair suture 100, 600 through the anterosuperior portal using a suture retriever. Insert a curved SutureLasso™ suture passer into the anteroinferior cannula and pass it through the capsulolabral tissue inferior to the anchor. Advance a nitinol wire loop 22 into the joint. Retrieve the wire loop 22 through the anterosuperior portal using a suture retriever.

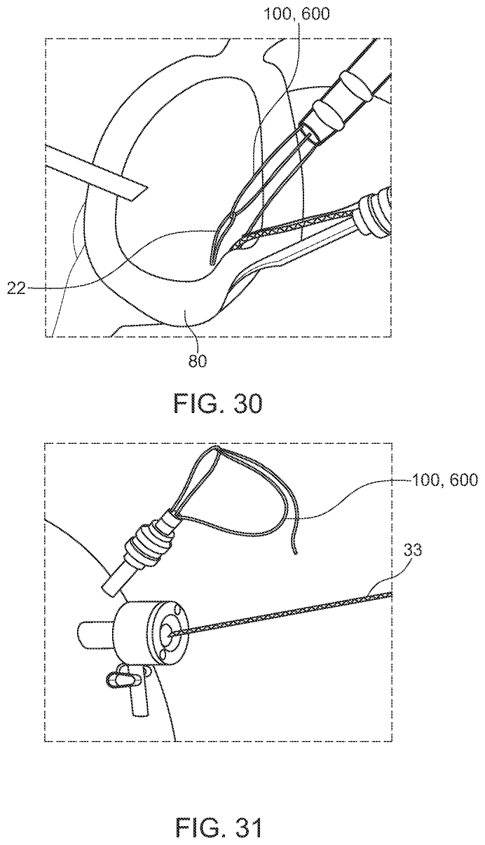

FIG. 30: Load the repair suture tail 100, 600 through the nitinol wire loop 22. Retract the wire loop through the SutureLasso™ suture passer to pull the suture to the distal end of the suture passer inside the joint. Remove the suture passer and wire loop together to shuttle the repair suture through the labral tissue.

FIG. 31: Retrieve the repair suture 100, 600 and shuttle suture 33 through the anterosuperior portal. Load the repair suture 100, 600 through the loop of the shuttle suture 33.

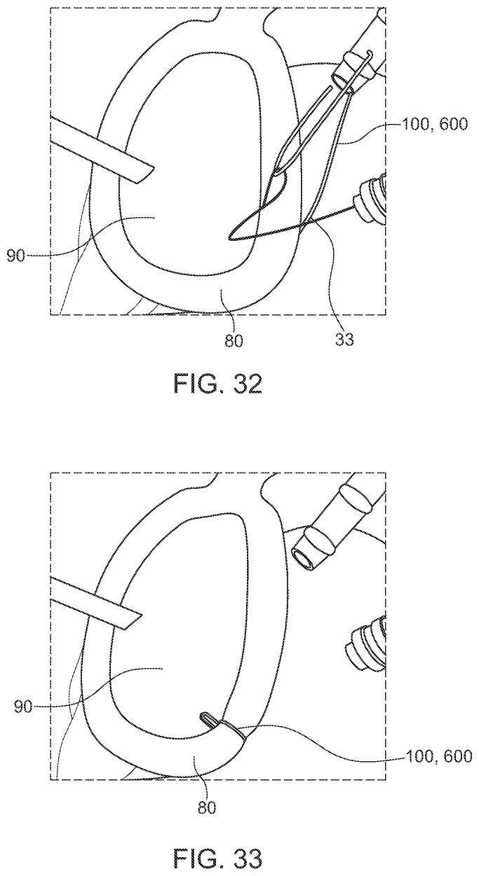

FIG. 32: Pull the shuttle suture 33 to transfer the repair suture 100, 600 back into the anchor body 333 through the same portal where it was inserted. Advance the shuttle suture 33 until the repair suture 100, 600 is passed through the suture splice locking mechanism and back out of the cannula.

FIG. 33: Pull the free end of the repair suture 100, 600 until the desired repair tension is achieved. A tissue grasper can be used to position the labrum in its desired location while applying tension on the repair.

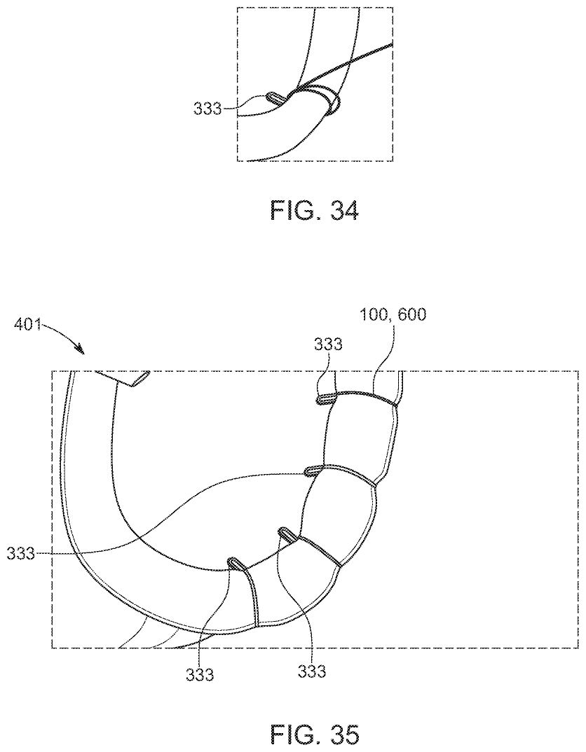

FIGS. 34 and 35 illustrate final labrum repair 401.

### Fraction Reduction and Fixation Using Cerclage

The embodiment below will be described with reference to elastic cerclage suture in the form of elastic suture tape such as elastic FiberTape® suture tape. The elastic FiberTape® suture tape cerclage system is an all-suture alternative to metal wires and cables traditionally used for circumferential fracture fixation. Fracture Reduction and Fixation with superior results can be achieved using elastic FiberTape® suture tape cerclage technology, and as detailed below. Elastic FiberTape® suture tape can be any FiberTape® suture tape modified with an elastic component, for example, elastic flexible coupler 300, 400, 600. An exemplary elastic FiberTape® suture tape can be an elastic 2 mm FiberTape® cerclage suture.

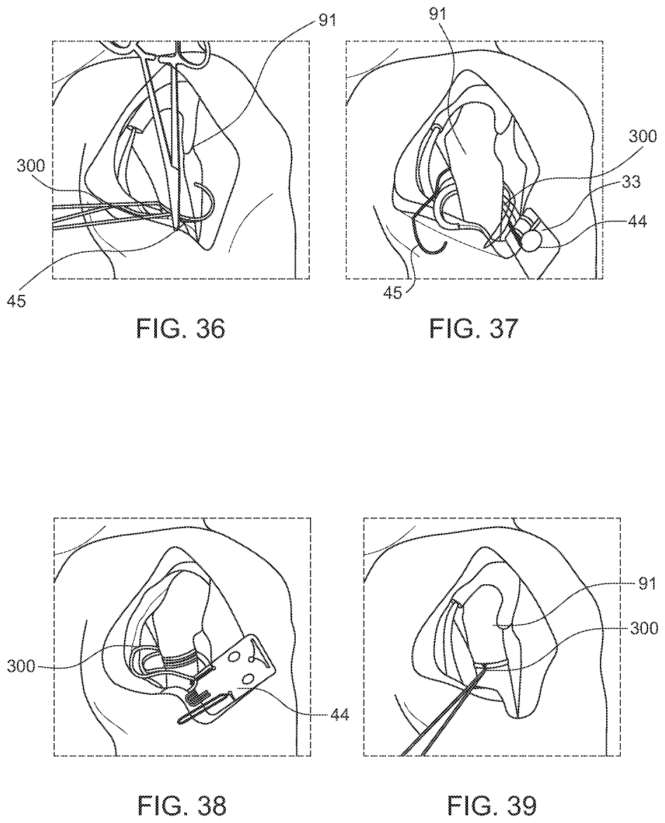

### FIGS. 36-45: Elastic FiberTape® Suture Tape Cerclage

Elastic cerclage suture (elastic FiberTape® suture tape) can be manufactured and packaged (can come configured) in a card 44 with a pretied knot and shuttle suture 33 (as shown, for example, in FIGS. 37 and 38).

FIG. 36: Load the tail of the cerclage suture 300 through an eyelet of needle 45. A passing hook can be employed in lieu of the needle. Pass the needle or passing hook around humerus 91.

FIG. 37: Load the tail of the cerclage suture 300 into the suture shuttle on the needle. Shuttle the cerclage suture 300 around the humerus 91 a second time.

FIG. 38: Load the tail of the cerclage suture 300 through the suture shuttle 33 (#1 on the card). Hold the card at the bullseye (#2 on the card) and shuttle the cerclage suture 300 through the pretied knot by pulling on the opposite loop (#3 on the card). Remove the card 44 and discard the suture shuttle 33.

FIG. 39: Reduce the knot close to the humerus 91 and remove the slack from the cerclage loops.

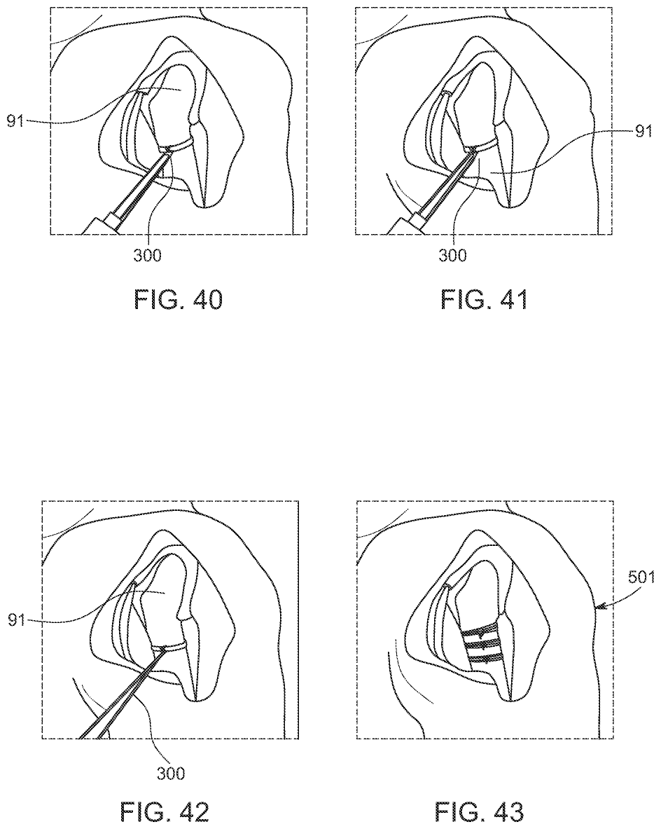

FIG. 40: Cut the tape 300 so there are two limbs. Place the limbs into a tensioner.

FIG. 41: Place the tensioner against the knot and begin to tension the tapes. Tension until the slack is removed from the loops.

FIG. 42: Press the release button on the tensioner and remove the suture limbs. Tie one half-hitch. Reload the suture limbs into the tensioner and conduct a final tension. Tie alternating half-hitches to complete the repair 501.

FIG. 43: Repeat the sequence for subsequent cerclage sutures of repair 501.

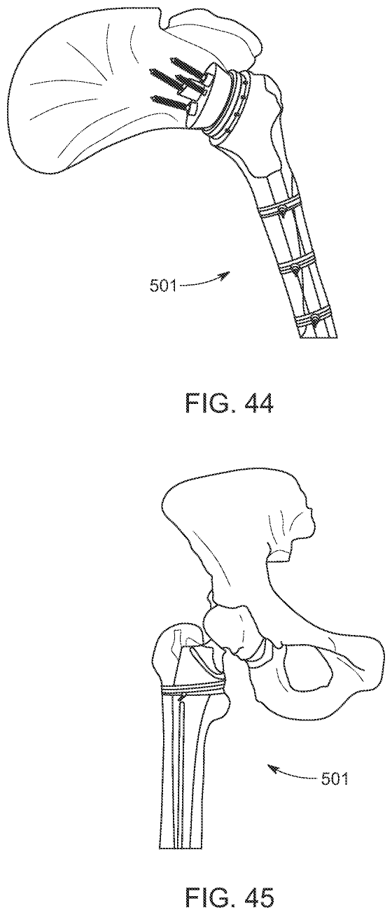

FIG. 44 illustrates exemplary elastic cerclage repair 501 described above employed with a revision-length humeral stem to provide a stronger repair following a periprosthetic fracture.

FIG. 45 illustrates exemplary elastic cerclage repair 501 described above employed prophylactically to help prevent intraoperative proximal femur fractures during hip arthoplasty.

### Racking Half Hitch

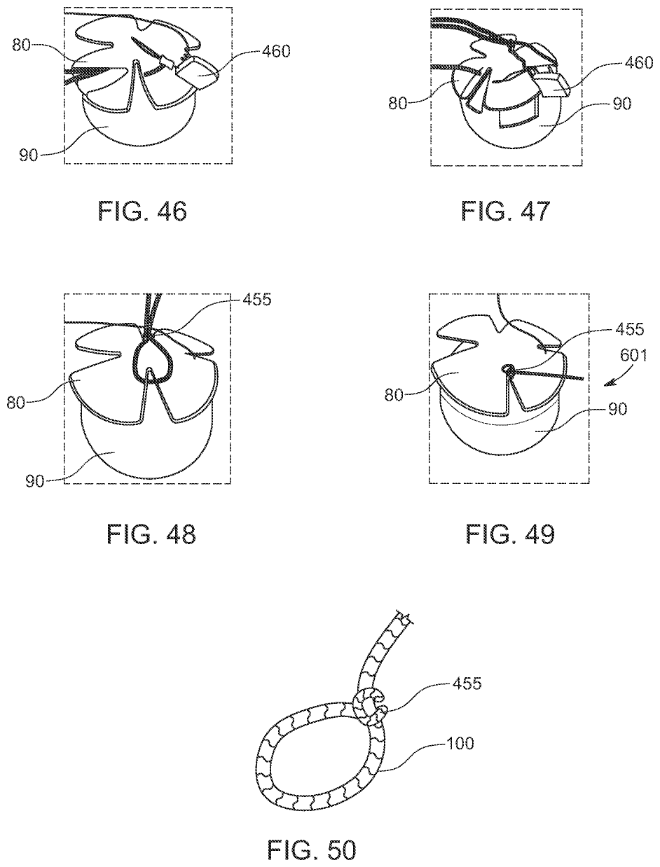

Reference is now made to FIGS. 46-50. A racking half hitch repair can be conducted with any of elastic suture 100, 200, 300, 400, 500, 600. As shown below, exemplary elastic suture 100 of repair 601 (FIG. 49) can be configured with a sheath that holds without the need to tie additional half hitches 455 to lock the racking hitch.

The jacket of the elastic suture is designed to hold on the section where the racking half hitch knot 455 is located. The suture tails can be swedged together for easy passing. A suture passing flag/card/device 460 (FIGS. 46 and 47) can hold the racking half hitch open. The tail/tails can be passed through the racking half hitch with the passing device to complete the knot 455. The knot 455 is then tightened down to secure the tissue 80. The elastic suture stretches/narrows during tension/tightening and then relaxes to capture/secure in place.

The elastic structures, constructs, and assemblies of the present disclosure have applicability to various surgical techniques and tissue repairs where the “elastic/stretchy” suture replaces the current static suture used in the art. Exemplary only applications of the “elastic/stretchy” suture of the present disclosure are:

Any tensionable knotless anchor—the elastic/stretching component can be incorporated into the repair suture to act as a stuffing or a dynamic repair to allow for either a more secure locking mechanism or as not to strangle the repair area.

Any sheath for all (or any) suture anchor can be made with elastic/stretching suture to allow for a better bunching and fixation.

Any internal brace repair—the FiberTape® suture tape can be replaced with an elastic/stretching suture to allow for a dynamic repair and have the ability to move and rebound when necessary.

Any FiberTape® Cerclage construct—the entire suture or part of the suture can be made with the elastic/stretching suture to keep the knot secure.

Exemplary elastic sutures 100, 200, 300, 400, 500, 600 of the present disclosure can be manufactured as a one-piece suture construct. Exemplary elastic sutures 100, 200, 300, 400, 500, 600 can be securely engaged to and disengaged from one or more fixation devices, such as fixation device 60. The fixation device 60 can be a suture anchor that provides at least one anchoring point of a first tissue affixed to a second tissue, for example, soft tissue to bone. Exemplary elastic sutures 100, 200, 300, 400, 500, 600 can be attached to one or more fixation devices by sliding through at least a portion of the fixation device, for example, by extending/sliding through at least a portion of a body of the fixation device.

An exemplary fixation device can be an anchor (knotted anchor, knotless anchor, or all-suture anchor), implant, button, screw or any fixation device that confers secure attachment and fixation of soft tissue 80 over bone 90 and that allow sliding of elastic suture within a body of fixation device. The fixation device can be a knotless suture anchor such as a two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of both of which are fully incorporated by reference in their entirety herein.

Another exemplary fixation device is a soft anchor or an “all-suture” anchor. A soft anchor (soft suture anchor or all-suture soft knotless anchor) is provided with a soft anchor sleeve (sheath or tubular member) with two open ends, and at least two flexible shuttling strands extending through the soft anchor sleeve (sheath). The flexible strands may extend through the sleeve in similar or different directions and/or orientations and/or locations. The flexible tubular sleeve with the shuttling strands may be secured into or onto bone, and flexible strands may pass over soft tissue (rotator cuff) and are secured into bone to approximate soft tissue to bone. Details of an exemplary soft suture anchor with a soft anchor sleeve (sheath or tubular member) and flexible shuttling strands are set forth, for example, in U.S. Pat. No. 10,849,734 issued Dec. 1, 2020, entitled “Methods of Tissue Repairs,” the disclosure of which is incorporated by reference in its entirety herein.

The elastic flexible couplers, surgical assemblies and elastic constructs detailed above have applicability to any tissue repair and surgical procedure such as, for example, rotator cuff repairs, Achilles tendon repair, patellar tendon repair, ACL/PCL reconstruction, hip and shoulder reconstruction procedures, and applications for elastic suture used in or with suture anchors. The surgical elastic constructs and repair methods of the present disclosure can be employed in tissue repairs that do not involve knot tying, for example, for use with suture anchors (such as PushLock® and/or SwiveLock® suture anchors) or for knotless arthroscopic suture repairs (such as knotless single row rotator cuff repair, or SpeedBridge™ repairs using no knots and only suture passing steps), among many others.

An exemplary SutureBridge™ tendon repair technique, developed by Arthrex, Inc., and disclosed in U.S. Pat. No. 8,012,174 (the disclosure of which is herein incorporated by reference in its entirety) consists of a tied medial row constructed with two threaded suture anchors, combined with knotless lateral fixation using two Arthrex PushLocks® constructs. The construct enhances footprint compression and promotes tendon healing-to-bone with minimal knot tying.

An exemplary SpeedBridge™ technique, also developed by Arthrex, Inc., and disclosed in U.S. Pat. No. 9,005,246 (the entire disclosure of which is herein incorporated by reference) uses a threaded swivel anchor which can be combined with any elastic flexible coupler 100, 200, 300, 400, 500, 600 in lieu of or in addition to FiberTape® suture tape to create a quick and secure SutureBridge™ construct with no knots and only two suture passing steps.

Exemplary fixations devices 60 can be two exemplary Arthrex SwiveLock® anchors 60 (for example, Arthrex 4.75-mm BioComposite SwiveLock® anchors) fixed to bone 90, 91 (such as humerus 91). Fixation devices 60 can be employed to fixate soft tissue to bone by a SpeedFix™ or SpeedBridge™ configuration. The SpeedFix™ and SpeedBridge™ techniques, both developed by Arthrex, Inc., use a threaded swivel anchor, such as Arthrex SwiveLock® C anchor combined with FiberTape® (disclosed in U.S. Pat. No. 7,892,256) to create a quick and secure SpeedFix™ construct (a knotless single row repair) or a SpeedBridge™ construct (a knotless double row repair) with no knots and very few suture passing steps.

In the SpeedFix™ technique, FiberTape® suture is passed in an inverted mattress using a SutureLasso™ or Scorpion™ suture passer. The two suture limbs of the mattress stitch can then be inserted into the SwiveLock® anchor eyelet. The loaded eyelet is inserted into a prepared lateral bone socket until the anchor body contacts bone, and the tension is adjusted if necessary. The SwiveLock® C driver is rotated in a clockwise direction to complete the insertion. Using an open ended FiberWire® cutter, the FiberTape® tails are cut to complete the technique.

A surgical assembly for tissue reinforcement and/or repair comprises an elastic flexible coupler 100, 200, 300, 400, 500, 600; and at least one fixation device 60 attached to the flexible coupler 100, 200, 300, 400, 500, 600. The fixation device 60 can be a knotless suture anchor. The tissue can be rotator cuff. The fixation device 60 can be a hard body anchor. The fixation device 60 can be a soft anchor.

A method of soft tissue reconstruction comprises attaching soft tissue 80 to bone 90, 91 with at least one elastic flexible coupler 100, 200, 300, 400, 500, 600. The method can further comprise: slidingly attaching the at least one elastic flexible coupler 100, 200, 300, 400, 500, 600 to a fixation device 60; and securing the fixation device 60 to bone 90, 91. The at least one elastic flexible coupler 100, 200, 300, 400, 500, 600 can be a one-piece construct consisting essentially of elastic suture. The fixation device 60 can be a knotless anchor.

Elastic flexible couplers 100, 200, 300, 400, 500, 600 can comprise suture tape such as FiberTape® suture tape (as disclosed in U.S. Pat. No. 7,892,256, the disclosure of which is incorporated in its entirety herewith) or collagen tape, or wide “tape like” material, or combinations thereof.

Elastic flexible couplers 100, 200, 300, 400, 500, 600 can be formed of a high strength suture material such as FiberWire® suture, sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided with at least one other elastic fiber, natural or synthetic, to form lengths of suture material.

Elastic flexible couplers 100, 200, 300, 400, 500, 600 can consist essentially of suture material and elastane, or combination of suture material and elastane and other materials such as long chain synthetic polymers like polyester and nylon, or materials such as PET, silk nylon or absorbable polymers, or coating materials (such as wax, silk, or silicone products), among many others. Elastic flexible couplers 100, 200, 300, 400, 500, 600 can consist of strands with cross-sections of various forms and geometries, including round, oval, rectangular, or flat, among others, or combinations of such forms and geometries. In an embodiment, at least one of elastic flexible couplers 100, 200, 300, 400, 500, 600 can be provided as a suture which is braided, knitted or woven.

Elastic flexible couplers 100, 200, 300, 400, 500, 600 can be also coated and/or provided in different colors. In an embodiment, parts (or all) of elastic flexible couplers 100, 200, 300, 400, 500, 600 can be coated (partially or totally) with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon. Hostaflon, or others), PBA (polybutylate acid), ethyl cellulose (Filodel) or other coatings, to improve lubricity of the suture and/or tape, pliability, handleability or abrasion resistance, for example.

Elastic flexible couplers 100, 200, 300, 400, 500, 600 can be also provided with tinted tracing strands, or otherwise contrast visually with other parts of the construct, which remain a plain, solid color, or displays a different tracing pattern, for example. Various structural elements of the surgical constructs may be visually coded, making identification and handling of the suture legs simpler. Easy identification of suture in situ is advantageous in surgical procedures.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. An elastic suture comprising:
an elastic core; and
a tubular jacket covering the elastic core, wherein the tubular jacket consists essentially of polyester and UHMWPE.

2. The elastic suture of claim 1, wherein the elastic core consists essentially of elastane.

3. The elastic suture of claim 1, wherein the elastic core comprises elastic and non-elastic material.

4. The elastic suture of claim 1, wherein the tubular jacket is elastic.

5. The elastic suture of claim 1, wherein the tubular jacket is non-elastic.

6. The elastic suture of claim 1, wherein the elastic suture is braided, weaved, or knitted.

7. The elastic suture of claim 1, wherein the elastic suture is manufactured as a one-piece construct.

8. The elastic suture of claim 1, wherein at least one of the elastic core and the tubular jacket comprises elastane.

9. The elastic suture of claim 1, wherein at least one region of the elastic suture is attached to a fixation device.

10. The elastic suture of claim 9, wherein the fixation device is a knotless suture anchor.

11. A suture tape comprising:
an elastic flat tape;
two elastic round tails extending about parallel to each other and within a body of the elastic flat tape; and
a non-elastic round tail.

12. The suture tape of claim 11, wherein the two elastic round tails and the non-elastic round tail are linked together to form a single tubular elastic tail.

13. A surgical assembly for soft tissue repairs, comprising an elastic flexible coupler attached to a knotless fixation device.

14. The surgical assembly of claim 13, wherein the elastic flexible coupler is an elastic suture comprising a silicon core and a jacket covering the silicon core, wherein the jacket consists essentially of a polyester and UHMWPE, and wherein the knotless fixation device is a knotless suture anchor.

15. The surgical assembly of claim 13, wherein the knotless fixation device is an anchor inserted into bone.

16. A method of soft tissue reconstruction comprising attaching soft tissue to bone with at least one elastic flexible coupler.

17. The method of claim 16, further comprising:
attaching the at least one elastic flexible coupler to a fixation device; and
securing the fixation device to the bone.

18. The method of claim 17, wherein the at least one elastic flexible coupler is elastic suture.

19. The method of claim 18, wherein the elastic suture includes a jacket covering an elastic core, wherein the jacket consists essentially of polyester and UHMWPE.

20. The method of claim 19, wherein the elastic core consists essentially of elastane material.

21. The method of claim 17, wherein the fixation device is a knotless anchor.

22. The method of claim 17, wherein the soft tissue is rotator cuff and the fixation device is a lateral or medial fixation device for rotator cuff repair.
